(19) United States
(12) Patent Application Publication
(10) Pub. No.: US 2026/0345152 A1
(43) Pub. Date: Nov. 5, 2026

(54) SUTURE MANAGEMENT DEVICES, SYSTEMS, AND METHODS

(71) Applicant: Boston Scientific Scimed, Inc., Maple Grove, MN (US)

(72) Inventors: Jason Nitin Mehta, Austin, TX (US); Alexander R. Cohen, Austin, TX (US); Timothy Noah Foster-Frau, Austin, TX (US); Nasser A. Ahmad, Austin, TX (US)

(73) Assignee: Boston Scientific Scimed, Inc., Maple Grove, MN (US)

(21) Appl. No.: 19/662,324

(22) Filed: Apr. 29, 2026

Related U.S. Application Data
(60) Provisional application No. 63/797,491, filed on Apr. 30, 2025.

(51) Int. Cl. A61B 17/04

(52) U.S. Cl. CPC A61B 17/0469; A61B 17/0401; A61B 2017/0409

(57) ABSTRACT

Devices, systems, and methods for managing a suture during use in a medical procedure. The length of suture extending away from the patient and not needed in the procedure is wrapped around a rotatable reel. Sufficient tension is applied to the suture to maintain the suture on the reel, yet to allow the suture to be pulled off the reel as more suture is needed for the medical procedure. In some aspects, the tension is adjustable. The suture management device may be mounted with respect to a medical device to be used in performing the medical procedure. One or more implantable medical devices may be included in the system and optionally mounted, with the suture management device, with respect to the medical device. The suture may extend along and be operably engaged with the implantable medical devices in a generally linear path to reduce friction on the suture.

### CROSS-REFERENCE TO RELATED APPLICATIONS

This application claims the benefit of priority under 35 U.S.C. § 119 to U.S. Provisional Application No. 63/797,491, filed on Apr. 30, 2025, the entire disclosure of which is hereby incorporated by reference herein for all purposes.

### FIELD

The present disclosure relates generally to the field of medical devices, systems, and methods using a suture or other flexible elongate element during a medical procedure. More particularly, the present disclosure relates to medical devices, systems, and methods for management of a portion of a suture remaining outside a body and before use during a medical procedure. The present disclosure further relates generally to the field of implantable medical devices, systems, and method usable with a suture. More particularly, the present disclosure relates to management of and facilitation of access to and use of more than one implantable medical device during a medical procedure, including management of tension on a suture operably associated with the implantable medical devices.

### BACKGROUND

Suture management is highly important for any suture based procedure, such as, without limitation endoscopic procedures. Without proper suture management, long lengths of suture can become tangled outside the patient’s body (e.g., outside the medical device, such as outside a medical scope), and/or wrap around the medical instruments used to deploy the suture (which can interfere with use of the medical instrument, such as by locking the medical instrument within an endoscope working channel through which the suture is delivered into the patient’s body). Deployment of multiple medical devices through which a suture extends, such as anchors or tacks, adds further complications to the procedure. Each of the multiple medical devices may add frictional forces on the suture as the suture passes through or otherwise engages a component of the multiple medical devices.

It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In some aspects, a suture management device formed in accordance with various principles of the present disclosure includes an axle; and a spool rotatably mounted with respect to the axle and configured for a length of suture to be wrapped therearound to be in an organized configuration. In some aspects, the spool includes a first element and a second element movable with respect to each other to adjust tension on the suture as the suture is released from the spool for use during a medical procedure.

In some aspects, the distance between the first element and the second element is adjustable to adjust tension on the suture. In some aspects, the spool is formed from a substantially planar element, and the first element and the second element are tabs extending at an angle with respect to each other to define a trough in which the suture may be extended and wound around the spool. In some aspects, at least one of a dimension, a number, flexibility, or stiffness of the tabs is adjustable to adjust tension imparted by the tabs on the suture. In some aspects, the spool comprises a top part and a bottom part defining a trough in which the suture may be wound. In some aspects, the top part comprises the first element, and the bottom part comprises the second element. In some aspects, the top part comprises a flexible portion which flexes with respect to the bottom part to allow suture to exit from the trough to be released from the suture management device. In some aspects, the flexible portion comprises a ridge extending toward the bottom part, the suture extending across the ridge to exit the trough. In some aspects, at least one of a dimension, stiffness, surface finish of one or both of the top part, the bottom part, or the ridge is adjustable to adjust tension imparted on the suture as the suture exits the trough. In some aspects, at least one of the top part or the bottom part is contoured to form a tortuous pathway for the suture to extend across to exit from the trough to be released from the suture management device. In some aspects, a distance between the top part and the bottom part is adjustable to adjust tension imparted by the contoured at least one of the top part or the bottom part on the suture.

In some aspects, a suture management system formed in accordance with various principles of the present disclosure includes a suture management device mount configured to be mount with respect to a medical device configured for use in performing a medical procedure with respect to a patient; a post on the mount; a spool rotatably mounted on the post; and a suture wrapped around the spool and unwindable off the spool as used during the procedure.

In some aspects, the spool includes a first element and a second element movable with respect to each other to adjust tension on the suture as the suture is released from the spool for use during a medical procedure.

In some aspects, the suture management system further includes one or more implantable medical devices operably associated with the suture and mounted on a device mount mounted on the suture management device mount. In some aspects, the one or more implantable medical devices includes at least two implantable medical devices aligned along a longitudinal axis of the device mount for suture to extend longitudinally through the two or more implantable medical devices. In some aspects, the device mount includes a riser for each of the at least two implantable medical devices, each riser angled with respect to the longitudinal axis to facilitate access sequential access to each of the at least two implantable medical devices to release the at least two implantable medical devices from the device mount for use during the procedure.

In some aspects, a method of managing a length of suture extending outside a patient’s body during performance of a procedure with respect to the patient’s body using the suture includes, in accordance with various principles of the present disclosure, pulling the suture off a reel about which the suture has been wound; and adjusting tension on the suture, as needed, to control release of the suture from the reel.

In some aspects, the method further includes mounting a suture management device mount, on which the reel is mounted, to a device used in performing the procedure.

In some aspects, the suture management device mount includes a device mount on which two or more implantable medical devices are mounted, the method further including pulling the suture through the two or more implantable medical devices in an axial direction parallel to a longitudinal axis of the device mount.

In some aspects, the two or more implantable medical devices are each mounted at angle with respect to the longitudinal axis of the device mount to facilitate access thereto, the method further comprising accessing and removing each of the two or more implantable medical devices sequentially for use in the procedure.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an example of an embodiment of a suture management system formed in accordance with various principles of the present disclosure and mounted with respect to a medical delivery and/or deployment device.

FIG. 2 illustrates an example of an embodiment of a suture management system formed in accordance with various principles of the present disclosure.

FIG. 3 illustrates a side view of the suture management system of FIG. 2.

FIG. 4 illustrates an example of an embodiment of a suture management system formed in accordance with various principles of the present disclosure.

FIG. 5 illustrates a side view of the suture management system of FIG. 2.

FIG. 6 illustrates a cross-sectional view along line VI-VI of FIG. 4.

FIG. 7 illustrates an example of an embodiment of a suture management system formed in accordance with various principles of the present disclosure.

FIG. 8 illustrates a side view of the suture management system of FIG. 7.

FIG. 9A illustrates a cross-sectional view along line IX-IX of FIG. 7.

FIG. 9B illustrates a cross-sectional view similar to that of FIG. 9A, but in a modified configuration.

FIG. 10 illustrates an exploded view of an implantable medical device and suture management system.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

Various medical procedures utilize a flexible elongate element to join together medical devices and/or medical device components and/or anatomical structures. The flexible elongate element may be a suture or other flexible elongate element known to those of ordinary skill in the art, the present disclosure not be limited in this regard.

In accordance with various principles of the present disclosure, a suture management device and associated system are configured to maintain a length of suture (referenced herein as simply a suture, for the sake of convenience and without intent to limit) in an organized, managed configuration during a procedure so that the suture does not tangle outside or inside the patient (e.g., around a medical instrument with which the suture is manipulated). In some aspects, a suture management device and associated system formed in accordance with various principles of the present disclosure provide controlled application of friction as a suture unravels, and retention of suture in such a way that it does not take a set. In some aspects, the suture management device and system provide the ability to wind and unwind the suture as needed during a procedure while maintaining the suture in an orderly arrangement.

In some aspects, a suture may be wrapped around a spool and/or axle to maintain the length of suture in a generally confined configuration. The spool and/or axle are configured to release the suture from the suture management device for use during a procedure. It will be appreciated that terms such as release, pay out, feed out, unwind, unravel, be pulled off, etc., including other grammatical forms thereof, may be usable interchangeably herein, without intent to limit. It will further be appreciated that terms such as spool, wheel, disk, reel, etc., may be usable interchangeably herein without intent to limit. Finally, it will be appreciated that terms such as axle, post, mount, pin, etc., may be usable interchangeably herein without intent to limit.

In accordance with various principles of the present disclosure, tension on the suture can be controlled in a variety of manners. In some aspects, light tension is maintained on the suture to prevent the suture from unspooling unintentionally, yet keeping the suture in an organized, managed arrangement with respect to the suture management device. In some aspects, such tension is less than about 2-7 lb, and preferably at least about ½ lb. As may be appreciated, it is generally desirable for the spool to always be able to feed suture as needed during a procedure. Accordingly, the tension preferably is less than about 3 lb. In some aspects, at 3 lb of tension, the suture may start having undesirable affects when interacting with tool, such as elongation, friction in channel, and/or pulling on the tool. If there is too much tension or friction on the suture, the suture can deform or even break, and/or may affect (e.g., ruin) the driver. Conversely, if there is too little tension, the suture may essentially freewheel off the suture management device into and unwieldy length of suture which may tangle or ball up or otherwise not be readily manageable or usable during a procedure.

In some aspects, a suture management device or system formed in accordance with various principles of the present disclosure is configured to adjust or tune the frictional load or tension on the suture, such as before or during a procedure.

In some aspects, one or more devices are operably associated with a suture prior to insertion, or at least during the procedure in which the suture is used. For instance, two or more anchors or tacks may be preloaded on a suture prior to insertion into the patient’s body. As the medical professional moves a tack, with a suture tied to it, away from the suture management device / system, the tension on the suture must not be so great that a tack which has already been implanted with respect to tissue within the patient’s body is not ripped out of the tissue. Thus, in some aspects, the suture management device and system should generate sufficient slack to allow movement of implantable medical devices coupled to the suture (as well as the suture itself) to be readily moved as needed during a procedure, yet also generate sufficient tension to maintain sufficient tension so that the suture does not freewheel and spin off the suture management device in an uncontrolled manner In some aspects, reduction of friction through the implantable medical devices limits the risk of suture fraying or taking a set when passing the suture through the implantable medical devices. As may be appreciated, it is desirable to reduce fraying and suture set for variety of reasons. For instance, fraying of the suture can reduce the overall holding strength of the suture, which impacts use of the suture (e.g., for cinching), and thus should be minimized if not eliminated. Suture set can impact how well the suture flows with respect to (e.g., through an element of) the tacks before and after placement within the patient’s body, which can affect delivery, deployment, and/or effectiveness of approximation of the implantable medical devices once deployed (e.g., to draw together tissue with respect to which the implantable medical device have been implanted).

In some aspects, a mount is provided to mount the suture management device with respect to the medical delivery and/or deployment device used to deliver and/or manipulate the suture, and/or used to deliver and/or to deploy an implantable medical device within the patient’s body. In some aspects, the mount is configured and designed specifically with suture management in mind. In some aspects, the mount may be used improve device usability. In some aspects, the mount facilitates the creation of more physician-controlled tools. In some aspects, the mount and allow for implantable medical devices to be packaged and sold separately from the medical delivery and/or deployment device, such as a separate cartridge or kit. The arrangement of the suture management device mount with respect to the suture and associated respective suture management device

Various embodiments of suture management systems, devices, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments are generally designated with the same reference numbers increased by a multiple of 1000 and redundant description is generally omitted for the sake of brevity. Moreover, certain features in one embodiment may be used across different embodiments and are not necessarily individually labeled when appearing in different embodiments. In some instances, the same reference number may be used to indicate elements or features that are not identical in form, shape, structure, etc., yet which provide similar functions or benefits. Additional reference characters (such as letters, as opposed to numbers) may be used to differentiate similar elements or features from one another.

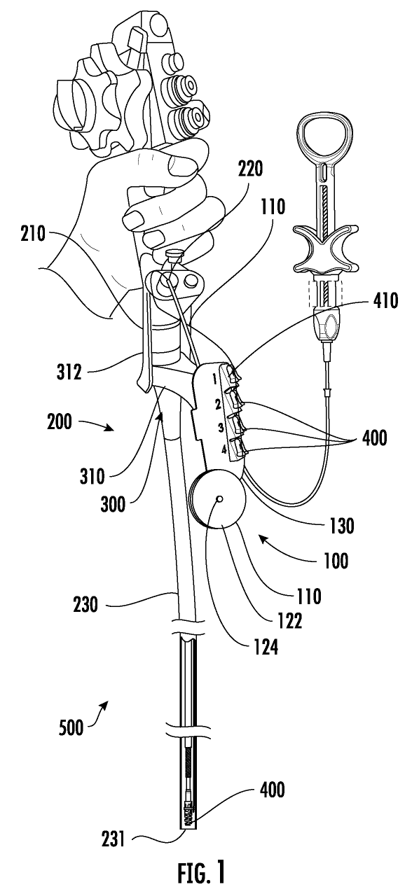

Turning now to the drawings, FIG. 1 illustrates an example of an embodiment of a suture management system 100 configured to manage a proximal portion of a suture 110 during a medical procedure in which the suture 110 is used. The suture management system 100 may be used in conjunction with a medical delivery and/or deployment device 200 which is configured to deliver the suture 110 into a patient’s body for use during a procedure performed with respect to the patient’s body. More particularly, the illustrated example of an embodiment of a medical delivery and/or deployment device 200 includes a control handle 210 defining a port 220 through which the suture 110 may be inserted into the patient’s body for use during a procedure within the patient’s body. Even more particularly, the illustrated example of an embodiment of a medical delivery and/or deployment device 200 includes a flexible tubular elongate member 230 which is sufficiently flexible to be navigated into curved or tortuous passages within a patient’s body (and may alternately be referenced as an insertion member) and which defines a lumen or working channel 231 through which devices, such as the suture 110, may be delivered into the patient. The port 220 is in operable communication with the working channel 231 so that devices are insertable into the working channel 231 via the port 220. In some aspects, the medical delivery and/or deployment device 200 is a medical scope, such as an endoscope, gastroscope, colonoscope, duodenoscope, laparoscope, ureteroscope, arthroscope, cystoscope, bronchoscope, hysteroscope, etc. An additional medical device may be used in conjunction with the medical delivery and/or deployment device 200 to deliver, use, and/or otherwise manipulate the suture 110 during a procedure. The additional medical device may be inserted into the port 220 and the working channel 231 to be delivered into a patient with the suture 110. Such medical device may be any of a variety of medical devices, instruments, tools, accessories, etc. (such terms being usable interchangeably herein without intent to limit) as known to those of ordinary skill in the art, the present disclosure not being limited in this regard.

In some aspects, the suture management system 100 may be mounted on or otherwise operably coupled with respect to the medical delivery and/or deployment device 200, such as to be conveniently accessible during a procedure. In the example of an embodiment illustrated in FIG. 1, a mounting system 300 may be used to mount or otherwise operably couple the suture management system 100 with respect to the medical delivery and/or deployment device 200, such as with respect to the control handle 210 of the medical delivery and/or deployment device 200. Such operable coupling may allow for subsequent removability of the mounting system 300, such as after the procedure has been performed with the suture 110. The mounting system 300 may be any configuration known to those of ordinary skill in the art for securing a medical device to another medical device. For instance, the mounting system 300 may include a mounting bracket 310 which may be configured to be engaged with the control handle 210 of the medical delivery and/or deployment device 200, such as by being snapped on or otherwise operably coupled or secured to the medical delivery and/or deployment device 200 (e.g., with respect to the control handle 210) to engage and be held in place with respect to the medical delivery and/or deployment device 200. In some aspects, further securement devices 312 may be used to secure the mounting bracket 310 with respect to the medical delivery and/or deployment device 200. For instance, the securement device 312 may be a strap or band or buckle which is wrapped around the medical delivery and/or deployment device 200 (e.g., the control handle 210 thereof), with at least one end thereof secured to the mounting bracket 310 and/or the medical delivery and/or deployment device 200.

In some aspects, the suture 110 is used in conjunction with one or more implantable medical devices 400. In some aspects, the implantable medical devices 400 may be operably associated with (e.g., mounted on or otherwise operably coupled with) the suture management system 100 for operable association with the suture 110 as well as with the medical delivery and/or deployment device 200. Such arrangement may facilitate access to and use of the implantable medical devices 400, such as with and during use of the suture 110 and the suture management system 100 of the present disclosure. In some aspects, the implantable medical devices 400 are arranged with respect to one another and/or the suture management system 100 to facilitate use of the implantable medical devices 400 and/or the suture 110 during a procedure, as will be described in further detail below. It will be appreciated that the various aspects of the implantable medical devices 400 and arrangement thereof with respect to one another and/or a suture 110 described herein may be implemented separately from the various aspects of the suture management system 100 described herein. Likewise, it will be appreciated that the various aspects of the suture management system 100 described herein may be implemented with or without the various aspects of the implantable medical devices 400 described herein.

In some aspects, the suture management system 100 and the medical delivery and/or deployment device 200, and optionally the suture 110 and/or the mounting system 300 and/or the implantable medical devices 400 (and associated components) as well, may together be considered a medical procedure system 500.

The example of an embodiment of a suture management system 100 illustrated in FIG. 1 includes a generically illustrated suture management device 120. A suture management device 120 formed in accordance with various principles of the present disclosure includes a spool 122 around which the suture 110 is wound. The spool 122 is rotatably mounted on an axle 124 so that the spool 122 rotates to pay out or feed or dispense or unwind or unravel or otherwise allow the suture 110 to be unwound or released or pulled off from the suture management device 120 for use during a procedure. Rotation of the spool 122 is set or regulated so as not to create too much resistant to release of the suture 110 therefrom, yet to provide sufficient resistance to maintain controlled release of the suture from the spool 122, such as may be determined by one of ordinary skill in the art, such as based on the suture material, devices used to manipulate the suture and/or to be delivered with the suture, the procedure in which the suture is being used, the nature of the anatomical structure with which the suture is being used, etc. In some aspects, the radius of curvature of the spool 122 is selected so that the suture 110 does not take a set after manufacture and prior to use (which may occur if the radius of curvature is too tight). To facilitate use during a procedure, the suture management system 100 may include a suture management device mount 130 which is configured to be operably associated with the above-described mounting system 300, such as with respect to the above-described mounting bracket 310. The suture management device mount 130 may be a simple platform, such as planar card, such as a die card (paperboard or plastic or other laminate structure) or any other structure on which the axle 124 of the suture management device 120 may be mounted so that spool 122 may be rotatably mounted with respect to the suture management device mount 130.

In accordance with various principles of the present disclosure, the suture management device 120 includes one or more structures or features which control or regulate the tension on the suture 110 as it is released from the suture management device 120, as described with reference to the examples of embodiments of suture management systems illustrated in FIGS. 2-8, 9A and 9B. For instance, one or more structures of the spool 122 may be positioned to regulate release of the suture 110 and/or to apply appropriate tension on the suture 110 so that the suture 110 may be pulled to the treatment site as needed and with a force appropriate for the medical professional performing the procedure as well as for the integrity of the suture 110 (e.g., so that excess force need not be applied to the suture 110 to pull more suture 110 to the treatment site). In some aspects, the relative position of two or more structures with respect to which the suture 110 moves may be adjusted to control friction or tension on the suture 110.

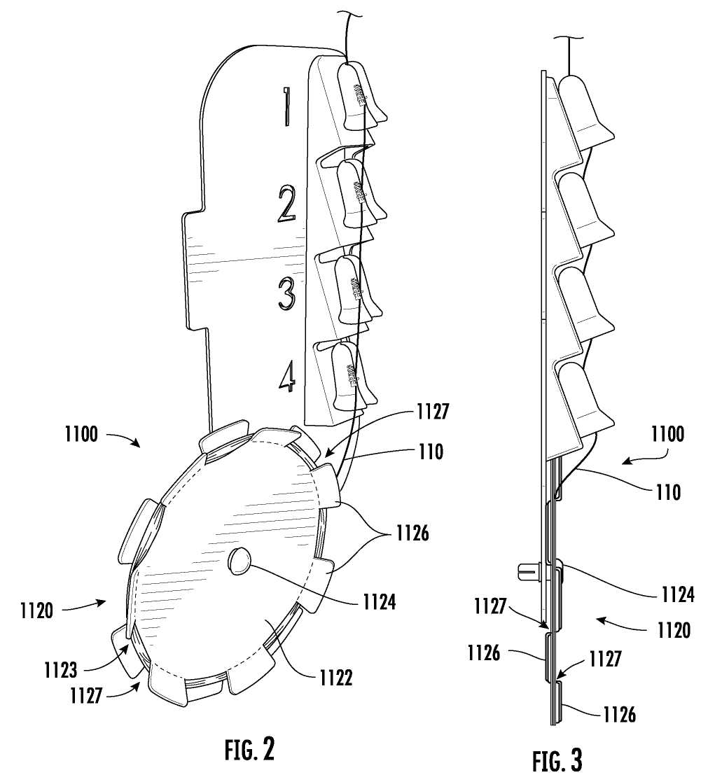

Turning to the example of an embodiment of a suture management system 1100 illustrated in FIGS. 2 and FIG. 3, the spool 1122 of the suture management device 1120 is formed from a flexible material, such as die cut paperboard or plastic material cut to form a plurality of attachment tabs 1126. In some aspects, the suture management device 1120 is formed of a substantially planar element. The attachment tabs 1126 may be formed by a plurality of spaced apart radially-extending slits 1127 along the periphery of the spool 1122. Adjacent attachment tabs 1126 may extend at angles with respect to each other (e.g., with respect to the plane of the spool 122 from which the attachment tabs 1126 extend) to define a trough 1123 in which the suture 110 may be extended and wound around the spool 1122. In some aspects, alternate ones of the attachment tabs 1126 may be moved out of the plane of the spool 1122 (in either axial direction along the axis of the axle 1124), as may be appreciated with reference to the side view of the suture management system 1100 illustrated in FIG. 3. The suture 110 may be wound about the periphery of the spool 1122 in the trough 1123 defined by and between attachment tabs 1126 extending in alternating directions, such as may be appreciated with reference to FIG. 3. As a medical professional performs a procedure using the suture 110, the medical professional pulls the suture 110 from the spool 1122, causing the spool 1122 to rotate about the axle 1124 and release suture 1110 from the spool 1122. The thickness and/or stiffness of the flexible material forming the attachment tabs 1126 can be selected to control how easily the suture releases from the attachment tabs 1126 and thus from the spool 1122. Additionally or alternatively, the number of attachment tabs 1126 may be selected to control the tension on the suture 110. Additionally or alternatively, the flexure of the attachment tabs 1126 with respect to the remaining unflexed portion of the spool 2122 may be selected to control the tension on the suture 110. Additionally or alternatively, the stiffness of the attachment tabs 1126 may be controlled by the length of the slits 1127 defining the attachment tabs 1126. For instance, larger (radially longer) slits 1127 would reduce stiffness and, as a result, tension on the suture 110, whereas smaller (radially shorter) slits 1127 would increase stiffness and, as a result, tension on the suture 110. Additionally or alternatively, the fitting of the spool 1122 with respect to the axle 1124 may be adjusted to adjust the rotatability of the spool 1122 with respect to the axle 1124 to thereby affect the tension the medical professional must apply on the suture 110 to pull the suture 110 off the spool 1122.

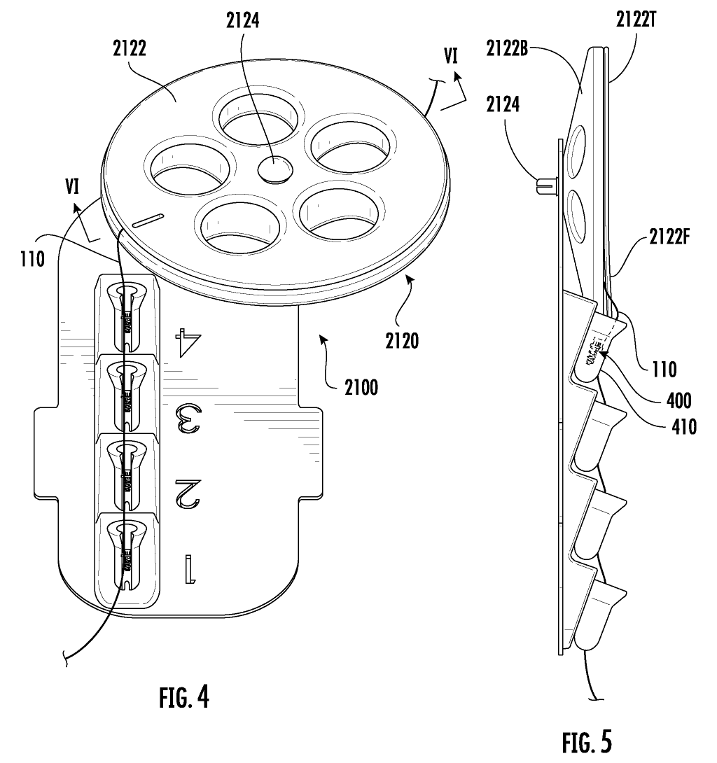

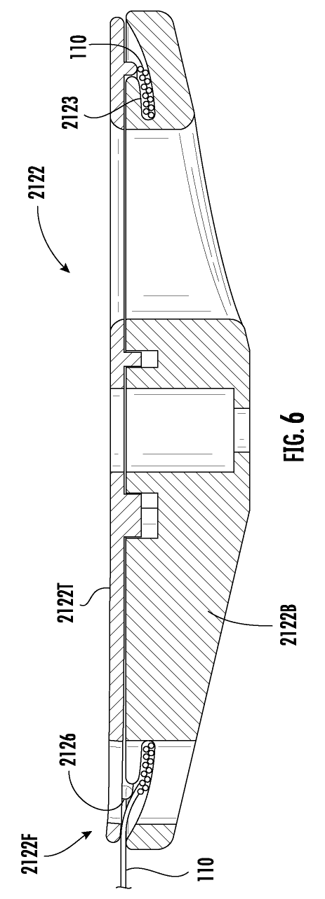

In the alternate example of an embodiment of a suture management system 2100 illustrated in FIGS. 4, 5, and 6, the suture management device 2120 includes a two-piece spool 2122 rotatable about an axle 2124. In accordance with various principles of the present disclosure, the two-piece spool 2122 is formed and configured to regulate tension on a suture 110. As may be appreciated with reference to the side view illustrated in FIG. 5, the top part 2122T of the two-piece spool 2122 includes a flexible portion 2122F capable of flexing away from the bottom part 2122B of the two-piece spool 2122. The suture 110 is stored / wrapped within a trough 2123 defined within the spool 2122, as illustrated in FIG. 6, along the cross-section line VI-VI in FIG. 4 (omitting the axle 2124). Flexure of the flexible portion 2122F of the top part 2122T of the spool 2122 with respect to the bottom part 2122B of the spool 2122 allows the suture 110 to extend out of the trough 2123 for use by the medical professional, such as at a treatment site. In some aspects, the suture 110 may extend out of its wound / stored configuration within the two-piece spool 2122 to one or more implantable medical devices 400, as illustrated in FIG. 5, and as discussed in further detail below. In some aspects, as may be appreciated with further reference to FIG. 6, the suture 110 is pulled off the spool 2122 over a ridge 2126 on the top part 2122T of the two-piece spool 2122. Various properties of top part 2122T and/or the ridge 2126, such as, without limitation, the height, material, surface finish, etc., may affect the rate at which the suture 110 may be pulled out of the two-piece spool 2122 at a reasonable force which does not put undue stress on the suture 110 and/or tissue with which the suture 110 is operably associated within the patient’s body. The flexible portion 2122B and/or the ridge 2126 may be limited to the flexible portion 2122B of the top part 2122T or may extend further along the top part 2122T, such as circumferentially around the top part 2122T. The bottom part 2122B of the two-piece spool 2122 may be formed to be more rigid than the top part 2122T to retain the suture 110 encapsulated under the ridge. The distance between the top part 2122Tand the bottom part 2122B; and/or the flexibility of the flexible portion 2122B of the top part 2122T; and/or the stiffness of the bottom part 2122B; and/or the dimensions (axial height, radial dimensions, etc.) of the ridge 2126, top part 2122T, and/or bottom part 2122B; and/or material properties (e.g., surface finish) of the ridge 2126, top part 2122T, and/or bottom part 2122B may be adjusted to determine the tension which must be applied to the suture 110 to be pulled off the spool 122.

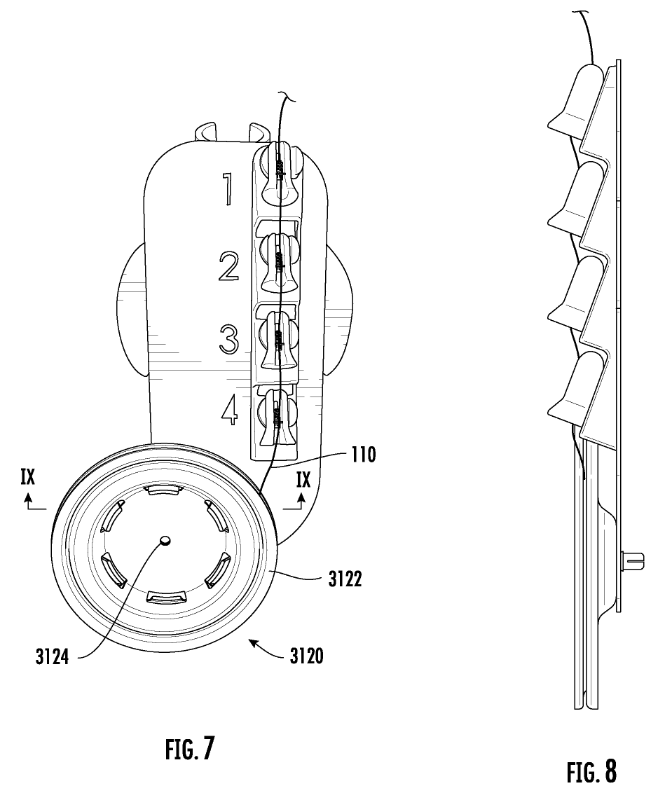

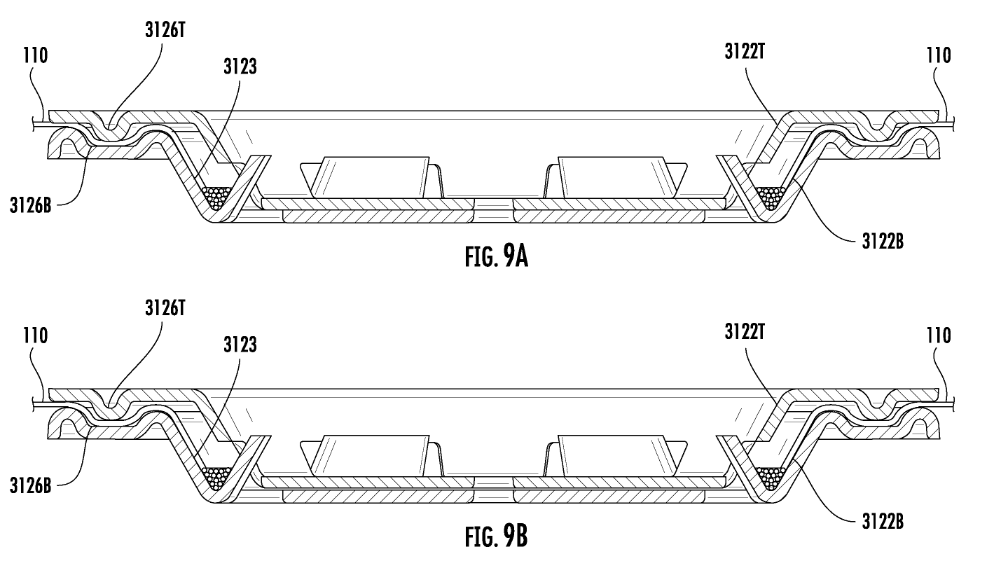

In the alternate example of an embodiment of a suture management system 3100 illustrated in FIGS. 7, 8, 9A and 9B, the suture management device 3120 includes two-piece spool 3122 rotatable about an axle 3124. In accordance with various principles of the present disclosure, the two-piece spool 3122 is formed with an internal geometry configured to regulate tension on a suture 110. In some aspects, such as illustrated in the cross-sectional view of FIG. 9A along the cross-section line IX-IX in FIG. 7, and in the cross-sectional view of FIG. 9B, showing an adjustment to the configuration illustrated in FIG. 9A (each cross-sectional view omitting the axle 3124) the two-piece spool 3122 is formed with a top part 3122T and a bottom part 3122B with inner surfaces which face each other and which have contours selected to create a tortuous path for the suture 110 which may be used to control friction and/or tension on the suture 110 to control release of the suture 110 from the two-piece spool 3122. For instance, the suture 110 may be wrapped within the two-piece spool 3122 within a trough 3123, around the axle 3124, and exit the space within the two-piece spool 3122 generally radially with respect to the axle 3124. As may be appreciated with reference to FIGS. 9A and 9B, ribs or channels or other geometries or surface contours or features 3126T, 3126B are formed in or along at least one of the top part 3122T and the bottom part 3122B of the two-piece spool 3122 to create a generally tortuous path along which the suture 110 radially extends out of the two-piece spool 3122. The amount of friction or tension applied to the suture 110 may depend on and/or be controlled by the geometry, materials, and/or surface characteristics of the top part 3122T and the bottom part 3122B of the two-piece spool 3122 along which the suture 110 extends. In some aspects, one or both of the top part 3122T and the bottom part 3122B of the two-piece spool 3122 are molded to define appropriate geometries for suture 110 retention and release. In some aspects, the two-piece spool 3122 may be configured to permit dynamic control of the spacing of the top part 3122T and the bottom part 3122B thereof. For instance, the top part 3122T and the bottom part 3122B of the two-piece spool 3122 may be threadedly coupled so that relative rotation of the top part 3122T and the bottom part 3122B (e.g., rotation of the top part 3122T relative to the bottom part 3122B which may be fixed to the above-described suture management device mount 130) controls the spacing between the features 3126T, 3126B defined by the inner surface of the top part 3122T and the bottom part 3122B (which face each other) to adjust (increase / decrease) friction and/or tension on the suture 110.

In one or more of the above-described examples of embodiments of suture management devices 120, 1120, 2120, 3120, the proximal free end of the suture 110 is not coupled to the suture management device 120, 1120, 2120, 3120 (e.g., the spool 122, 1122, 2122, 3122) so that the medical professional may readily access the proximal free end of the suture 110, such as to complete a procedure (e.g., by tying a knot in the suture 110 and/or applying a cinch lock or other type of locking element known to those of ordinary skill in the art to maintain a desired configuration, arrangement, etc., of a suture 110). In some aspects, the suture 110 is releasably coupled to the suture management device 120, 1120, 2120, 3120 (e.g., the spool 122, 1122, 2122, 3122). For instance, in the case of a two-piece spool 2122, 3122, the proximal end of the suture 110 may be pinched between the parts 2122T, 2122B, 3122T, 3122B of the two-piece spool 2122, 3122. Such releasable coupling may impart an initial restraint on the suture prior to release, and optionally an increase in friction as release of the end of the suture 110 is imminent. The change in force needed to release the suture 110 from the suture management device 120, 1120, 2120, 3120 (e.g., the spool 122, 1122, 2122, 3122) may provide a noticeable feedback to the user so the user does not pull the suture 110 off inadvertently. Such tactile notification to the user may be achieved by applying increased holding force (increased pinching or squeezing of the suture 110 within the spool 122, 1122, 2122, 3122) at the proximal free end of the suture 110 so the user does not accidentally pull out and lose control of the proximal free end of the suture 110.

In each of the examples of embodiments of suture management systems 100, 1100, 2100, 3100 illustrated in FIG. 1, one or more implantable medical devices 400 operably associated with the suture 110 managed by the illustrated suture management systems 100, 1100, 2100, 3100 may be mounted on the suture management device mount 130 on which the respective suture management device 120, 1120, 2120, 3120 is mounted. Such arrangement may be advantageous for facilitating access, use, reloading, etc., of the medical delivery and/or deployment device 200 with an implantable medical device 400, such as during a procedure. It will be appreciated that reference is made to the suture management device mount 130 illustrated in FIG. 1 which may be used in each of the of suture management systems 100, 1100, 2100, 3100, with or without variations among the suture management systems 100, 1100, 2100, 3100. The implantable medical devices 400 may thereby be operably associated with a medical delivery and/or deployment device 200 along with a suture management system 100, 1100, 2100, 3100, such as to provide easy access to the medical professional.

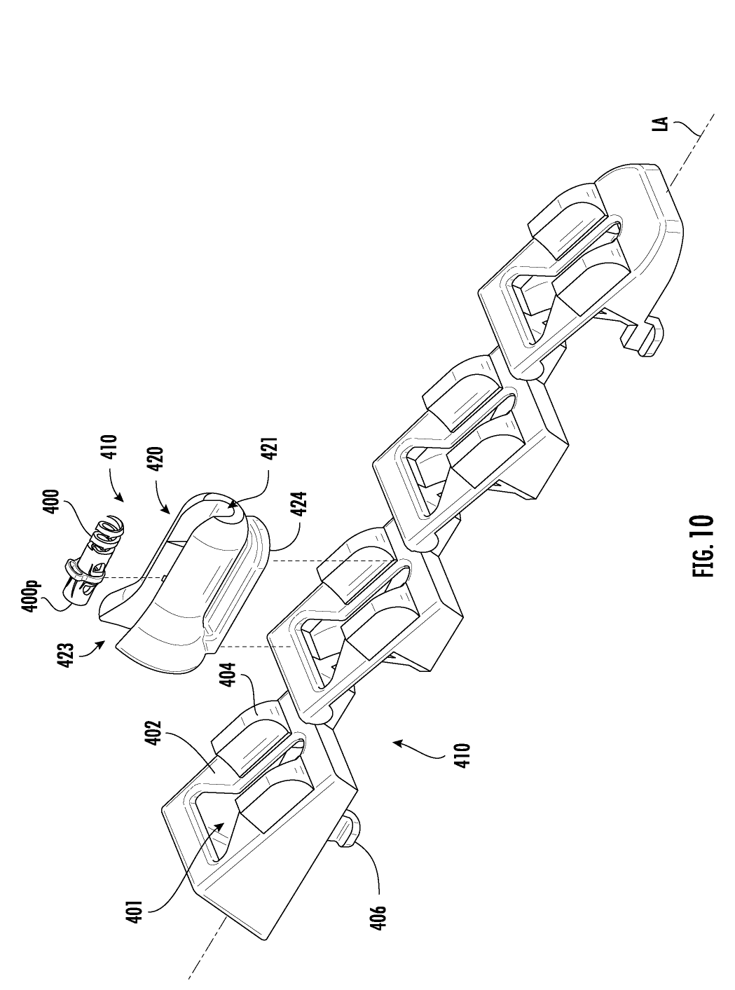

In the example of an embodiment of an implantable medical devices 400 illustrated in FIG. 1, the implantable medical devices 400 are anchors or tacks, as illustrated in further detail in FIG. 10. In accordance with various principles of the present disclosure, in order to manage (typically reduce) the amount of friction between the suture 110 and the implantable medical devices 400 through which the suture 110 extends, the implantable medical devices 400 may be substantially axially aligned with one another along the device mount 130. In some aspects, the implantable medical devices 400 are themselves mounted on a device mount 410 which is then operably coupled with the suture management device mount 130. In the example of an embodiment illustrated in FIG. 10, the device mount 410 is provided with a mounting seat 401 configured to receive a portion of an implantable medical device 400. The mounting seats 401 are axially aligned along the longitudinal axis LA of the device mount 410 so that a suture 110 may extend through each of the implantable medical devices 400 (e.g., through a portion of the implantable medical devices 400, such as an eyelet) in a substantially linear manner, parallel to the longitudinal axis LA of the device mount 410 (such as illustrated in FIG. 1), thereby reducing friction on the suture 110 as each of the implantable medical devices 400 is used with the suture 110 extending through the remaining unused implantable medical devices 400 and to the remaining extent of suture 110 not yet used (e.g., and optionally managed by any of the suture management systems 100, 1100, 2100, 3100 described above). As such, it may be appreciated that a device mount 410 formed in accordance with various principles of the present disclosure provides suture management benefits separate and independent of, yet optionally complementary to, the suture management benefits provided by the suture management systems 100, 1100, 2100, 3100 described above,

In the example of an embodiment of device mount 410 illustrated in FIG. 10, the device mount 410 includes a plurality of risers 402, such as one for each of the implantable medical device 400. In accordance with various principles of the present disclosure, one or more of the of risers 402 is angled with respect to the longitudinal axis LA to facilitate access to proximal end 400p of each implantable medical device 400 by the medical delivery and/or deployment device 200, such as for use in a procedure. As may be appreciated, the risers 402 may each define a mounting seat 401 for an implantable medical device 400 as described above. As may further be appreciated, the risers 402 may otherwise be aligned along the longitudinal axis LA of the device mount 410, such as described above, so that the suture 110 extends along implantable medical devices 400 mounted in the risers 402 in a direction parallel to the longitudinal axis LA.

In some aspects, the implantable medical devices 400 are relatively small, and are each individually mounted in a capsule 420 which is configured to retain the implantable medical device 400 therein, and mounting seats 401 the device mount 410 are configured to receive the capsules 420 which hold the implantable medical devices 400. In some aspects, the capsule 420 is formed of a flexible material such as silicone which facilitates retention in the mounting seat 401. In some aspects, a retainer arm 404 is provided on one or both sides of the device mount 410 (to grasp or otherwise retain the capsule 420, or the implantable medical device 400 if not within a capsule 420, with respect to the mounting seat 401 and device mount 410). However, it will be appreciated that the resiliency, grippiness, deformability, and/or other properties of the capsule 420 may sufficiently retain the capsule 420 with respect to the mounting seats 401 without the need for a retainer arm 404 (such as in the examples of embodiments illustrated in FIGS. 2, 3, 4, 5, 7, and 8). In some aspects, the capsule 420 includes a base 424 configured to form an interference fit with the mounting seat 401 so that a retainer arm 404 need not be provided.

In some aspects, the capsule 420 defines a capsule seat 421 configured to securely receive the implantable medical device 400 therein so that the implantable medical device 400 cannot be inadvertently separated from the capsule 420. In some aspects, a slit 423 facilitates intentional deformation of the capsule 420 in order to release the implantable medical device 400 therefrom for use, such as in a procedure.

In some aspects, the device mount 410 includes mounting structures 406 configured to mount the device mount 410 with respect to the suture management device mount 130. For instance, the mounting structure 406 may be in the form of projections or legs which may grasp the suture management device mount 130 and/or be inserted in apertures formed in the suture management device mount 130 to retain the device mount 410 with respect to the suture management device mount 130. In some aspects, the series of implantable medical devices 400 may be labeled, such as to indicate order of use (1, 2, 3, 4, etc.). In some aspects, indicia for labelling the implantable medical devices 400 may be provided on the suture management device mount 130, such as illustrated in FIGS. 1, 2, 4 and 7. As may be appreciated, the suture management device mount 130 may be oriented for ready viewing by the medical professional, such as illustrated in FIG. 4 (e.g., with the numbering substantially upside down in an elevational view, but appearing right-side up when viewed from above, as would be the case if mounted on a medical delivery and/or deployment device 200). In some aspects, the first implantable medical device 400 to be used is furthest from the suture management device 120, 1120, 2120, 3120.

It will be appreciated that an implantable medical device mount 410 as described above may be used with any of the above-described suture management systems 100, 1100, 2100, 3100 and/or suture management devices 120, 1120, 2120, 3120 described above, or independently (i.e., not operably associated with a suture management systems 100, 1100, 2100, 3100 or suture management devices 120, 1120, 2120, 3120).

It will be appreciated that the present disclosure describes separate and independent features and devices which may be used individually or in conjunction with one another. It will therefore be appreciated that although examples of embodiments are shown with a suture management systems 100, 1100, 2100, 3100 as well as an implantable device mount 410, each may be used separately and independently of the other, or in conjunction with each other, the present disclosure not being limited in this regard.

It should be therefore be understood that the various embodiments illustrated in the figures have several separate and independent features, which each, at least alone, has unique benefits which are desirable for, yet not critical to, the presently disclosed suture and implantable medical device systems. Therefore, the various separate features described herein need not all be present in order to achieve at least some of the desired characteristics and / or benefits described herein. Various further benefits of the various aspects, features, components, and structures of devices, systems, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and / or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader’s understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A suture management device comprising:
an axle; and
a spool rotatably mounted with respect to said axle and configured for a length of suture to be wrapped therearound to be in an organized configuration;
wherein said spool includes a first element and a second element movable with respect to each other to adjust tension on the suture as the suture is released from said spool for use during a medical procedure.

2. The suture management device of claim 1, wherein a distance between the first element and the second element is adjustable to adjust tension on the suture.

3. The suture management device of claim 1, wherein said spool is formed from a substantially planar element, and the first element and the second element are tabs extending at an angle with respect to each other to define a trough in which the suture may be extended and wound around said spool.

4. The suture management device of claim 3, wherein at least one of a dimension, a number, flexibility, or stiffness of said tabs is adjustable to adjust tension imparted by said tabs on the suture.

5. The suture management device of claim 2, wherein said spool comprises a top part and a bottom part defining a trough in which the suture may be wound.

6. The suture management device of claim 5, wherein said top part comprises said first element, and said bottom part comprises said second element.

7. The suture management device of claim 6, wherein said top part comprises a flexible portion which flexes with respect to said bottom part to allow suture to exit from the trough to be released from said suture management device.

8. The suture management device of claim 7, wherein said flexible portion comprises a ridge extending toward said bottom part, the suture extending across said ridge to exit the trough.

9. The suture management device of claim 8, wherein at least one of a dimension, stiffness, surface finish of one or both of said top part, said bottom part, or said ridge is adjustable to adjust tension imparted on the suture as the suture exits the trough.

10. The suture management device of claim 5, wherein at least one of said top part or said bottom part is contoured to form a tortuous pathway for the suture to extend across to exit from the trough to be released from said suture management device.

11. The suture management device of claim 10, wherein a distance between said top part and said bottom part is adjustable to adjust tension imparted by the contoured at least one of said top part or said bottom part on the suture.

12. A suture management system comprising:
a suture management device mount configured to be mount with respect to a medical device configured for use in performing a medical procedure with respect to a patient;
a post on said mount;
a spool rotatably mounted on said post; and
a suture wrapped around said spool and unwindable off said spool as used during the procedure.

13. The suture management system of claim 12, wherein said spool includes a first element and a second element movable with respect to each other to adjust tension on said suture as said suture is released from said spool for use during a medical procedure.

14. The suture management system of claim 12, further comprising one or more implantable medical devices operably associated with said suture and mounted on a device mount mounted on said suture management device mount.

15. The suture management system of claim 14, wherein said one or more implantable medical devices includes at least two implantable medical devices aligned along a longitudinal axis of said device mount for suture to extend longitudinally through said two or more implantable medical devices.

16. The suture management system of claim 15, wherein said device mount includes a riser for each of said at least two implantable medical devices, each riser angled with respect to said longitudinal axis to facilitate access sequential access to each of said at least two implantable medical devices to release the at least two implantable medical devices from said device mount for use during the procedure.

17. A method of managing a length of suture extending outside a patient’s body during performance of a procedure with respect to the patient’s body using the suture, said method comprising:
pulling the suture off a reel about which the suture has been wound; and
adjusting tension on the suture, as needed, to control release of the suture from the reel.

18. The method of claim 17, further comprising mounting a suture management device mount, on which the reel is mounted, to a device used in performing the procedure.

19. The method of claim 18, wherein the suture management device mount includes a device mount on which two or more implantable medical devices are mounted, said method further comprising pulling the suture through the two or more implantable medical devices in an axial direction parallel to a longitudinal axis of the device mount.

20. The method of claim 19, wherein the two or more implantable medical devices are each mounted at angle with respect to the longitudinal axis of the device mount to facilitate access thereto, said method further comprising accessing and removing each of the two or more implantable medical devices sequentially for use in the procedure.
